(19) United States
(12) Patent Application Publication
(10) Pub. No.: US 2009/0088578 A1
(43) Pub. Date: Apr. 2, 2009

(54) NUCLEAR MAGNETIC RESONANCE IMAGING OF SELECTIVE SMALL MOLECULE DRUGS AS CONTRAST AGENTS

(76) Inventors: Christopher D. Lascola, Durham, NC (US); Allen W. Song, Chapel Hill, NC (US); Timothy A. Haystead, Chapel Hill, NC (US)

Correspondence Address: JENKINS, WILSON, TAYLOR & HUNT, P. A., Suite 1200 UNIVERSITY TOWER, 3100 TOWER BLVD.,, DURHAM, NC, 27707, US

(21) Appl. No.: 12/017,608

(22) Filed: Jan. 22, 2008

Related U.S. Application Data
(60) Provisional application No. 60/881,423, filed on Jan. 19, 2007.

(51) Int. Cl. A61K 49/10; C07D 209/04; A61B 5/055

(52) U.S. Cl. 548/490; 424/9.3; 600/420

(57) ABSTRACT

Imaging agents for magnetic resonance imaging are disclosed. Also disclosed are methods of non-invasively generating a visible image of a target tissue. In some embodiments, the discloses methods include the steps of (a) providing a contrast enhancement agent comprising a small molecule that binds to a biomolecule present in the target tissue; (b) introducing the contrast enhancement agent into the target tissue; and (c) scanning the target tissue using magnetic resonance imaging, whereby a visible image of the target tissue is non-invasively generated. Further discloses are methods for monitoring a response to a therapy, methods for selecting a therapy for a subject, methods for delineating a boundary between a diseased cell and a non-diseased cell in a tissue, and methods for assessing the degree to which a target tissue has been removed from a subject.

### CROSS REFERENCE TO RELATED APPLICATION

This application claims the benefit of U.S. Provisional Patent Application Ser. No. 60/881,423, filed Jan. 19, 2007; the disclosure of which is incorporated herein by reference in its entirety.

### GRANT STATEMENT

This work was supported by Grant No. BES-0092672 from the National Science Foundation and Grant Nos. R01 NS50329, R01-MH 58251-01A3, RO1 DK 52378-09, RO1 HL 078795-01, P30 ES011961, and 2-P30-CA14238-3 from the United States National Institutes of Health. Accordingly, the United States government has certain rights in the presently disclosed subject matter.

### TECHNICAL FIELD

The presently disclosed subject matter relates to methods and compositions for performing magnetic resonance imaging (MRI). More particularly, the presently disclosed subject matter provides methods and compositions for magnetization transfer and/or saturation transfer MRI that can be employed for imaging small molecule interactions with macromolecules in vivo.

### BACKGROUND

Medical diagnostic imaging has evolved as an important non-invasive tool for the evaluation of pathological and physiological processes. These techniques have the advantage of allowing the physician to image regions of a subject's body without the need for surgical intervention, which is subject to many well-recognized drawbacks.

Currently employed non-invasive imaging techniques are not without their own limitations, however. In particular, these techniques typically require the use of imaging agents that must be administered to a subject prior to performing the imaging itself. Both the production and use of such imaging agents can limit the usefulness of the relevant techniques. For example, many strategies rely on the use of imaging agents that comprise gamma emitters, such as certain isotopes of iodine, or positron emitters, such as ¹⁸F or ¹¹C. In order to use these species in an imaging strategy, however, the species must be generated in a cyclotron, purified, complexed to the imaging agent, and administered, all before the imaging technique can be performed. Each of these isotopes has a half-life associated with it, some of which are very short. ¹⁸F, for example, has a half-life of only 110 minutes, and ¹¹C has a half-life of only 20 minutes. Thus, the use of these species involves technical hurdles that remain difficult to overcome.

Additionally, spatial resolution for non-invasive in vivo imaging remains poor. Positron emission tomography (PET), for example, has a spatial resolution of only about 2-4 mm in humans, which does not allow for imaging of anatomic detail to an acceptable degree in many applications.

Presently, nuclear magnetic resonance imaging (“MRI”) and computerized tomography (“CT”) are two of the most widely used imaging modalities. Although both MRI and CT can be performed without the administration of contrast agents, the ability of many contrast enhancement agents to enhance the visualization of internal tissues and organs has resulted in their widespread use.

These imaging modalities have several drawbacks, however. In the area of diagnostic imaging of cancer, for example, current methods for tumor-specific imaging include PET and SPECT. CT and MR contrast agents, on the other hand, are nonspecific in that they do not typically bind to biomarker targets (e.g., overexpressed genes/proteins), but simply extravasate where there are leaky blood vessels. These approaches are hindered by imaging agents that either cannot be delivered adequately to the target tissue and/or that accumulate in normal tissues. Particularly in the context of tumor imaging, while several genes have been found to be overexpressed in certain tumor types, these same genes are often expressed at a detectable level in non-tumor cells. This can greatly decrease the efficiency of imaging strategies that take advantage of imaging agents that are designed to target overexpressed gene products in tumors.

Additionally, a lack of targeting ligands that are capable of binding to multiple tumor types necessitates the synthesis of a wide range of agents in order to image different tumor types. Ideally, a targeting molecule should display an absence of substantial binding in normal tissues and a capacity for targeting to a variety of tumor types and stages.

Thus, there exists a long-felt need in the art for new methods and compositions that can be employed for non-invasive imaging of target tissues.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter provides contrast enhancement agents useful for providing a visible image of a biological sample. In some embodiments, the contrast enhancement agent comprises a small molecule ligand that binds to heat shock protein 90 (generically abbreviated as hsp90, although sometimes abbreviated HSP90 when referring specifically to the human gene or gene product). In some embodiments, the small molecule ligand comprises an indoline moiety. In some embodiments, at least one atom present in the contrast enhancement agent is a resonant nucleus. In some embodiments, the resonant nucleus is selected from the group including but not limited to ¹³C, ²³Na, ¹⁹F, ¹⁵N, ¹⁷O, and ³¹P.

In some embodiments, the presently disclosed contrast enhancement agents further comprise a pharmaceutically acceptable diluent or excipient. In some embodiments, the pharmaceutically acceptable diluent or excipient is pharmaceutically acceptable for use in a human.

The presently disclosed subject matter also provides imaging compositions comprising a small molecule ligand that binds to heat shock protein 90 (hsp90) and a pharmaceutically acceptable diluent or excipient. In some embodiments, the pharmaceutically acceptable diluent or excipient is pharmaceutically acceptable for use in a human.

The presently disclosed subject matter also provides methods for non-invasively generating an image of a target tissue. In some embodiments, the methods comprise (a) providing a contrast enhancement agent comprising a small molecule that binds to a biomolecule present in the target tissue; (b) introducing the contrast enhancement agent into the target tissue; and (c) scanning the target tissue using magnetic resonance imaging, whereby an image of the target tissue is non-invasively generated. In some embodiments, the imaging agent is disposed in a pharmaceutically acceptable diluent. In some embodiments, the target tissue comprises a plurality of cells that overexpress hsp90. In some embodiments, the target tissue is disposed in a subject. In some embodiments, the target tissue is selected from the group including but not limited to an inflammatory lesion, a tumor, a pre-neoplastic lesion, a neoplastic cell, a pre-neoplastic cell, and a cancer cell. In some embodiments, the tumor is selected from the group including but not limited to a primary tumor, a metastasized tumor, and a carcinoma. In some embodiments, the target tissue is selected from the group including but not limited to a prostate tumor, a breast tumor, a uterine tumor, a melanoma, a glioma, a bladder carcinoma, a laryngeal carcinoma, a salivary gland tumor, and a leukemia. In some embodiments, the magnetic resonance imaging comprises magnetization/saturation transfer MRI. In some embodiments, the biomolecule is an hsp90 polypeptide. In some embodiments, the hsp90 polypeptide is a human hsp90 polypeptide. In some embodiments, the introducing is by a route selected from the group including but not limited to oral, peroral, buccal, enteral, pulmonary, rectal, vaginal, nasal, lingual, sublingual, intravenous, intraarterial, intracardial, intramuscular, intraperitoneal, transdermal, intracranial, intracutaneous, subcutaneous, ocular, via an implant, and via a depot injection.

The presently disclosed subject matter also provides methods for monitoring a response to a therapy in a target tissue in a subject. In some embodiments, the methods comprise comparing a plurality of MRI images of a target tissue in the subject, wherein (a) a first subset of the plurality of MRI images are images of the target tissue generated prior to administering the therapy to the subject; and (b) a second subset of the plurality of MRI images are images of the target tissue generated at a time subsequent to administering the therapy to the subject and at which a response to the therapy is expected; and further wherein the plurality of MRI images are generated by a method comprising (i) providing a contrast enhancement agent comprising a small molecule that binds to a biomolecule present in the target tissue; (ii) introducing the contrast enhancement agent into the target tissue; and (iii) scanning the target tissue using magnetic resonance imaging, whereby a visible image of the target tissue is non-invasively generated. In some embodiments, the target tissue comprises a plurality of cells that overexpress hsp90. In some embodiments, the target tissue is disposed in a subject. In some embodiments, the target tissue is selected from the group including but not limited to an inflammatory lesion, a tumor, a pre-neoplastic lesion, a neoplastic cell, a pre-neoplastic cell, and a cancer cell. In some embodiments, the tumor is selected from the group including but not limited to a primary tumor, a metastasized tumor, and a carcinoma. In some embodiments, the target tissue is selected from the group including but not limited to a prostate tumor, a breast tumor, a uterine tumor, a melanoma, a glioma, a bladder carcinoma, a laryngeal carcinoma, a salivary gland tumor, and a leukemia. In some embodiments, the magnetic resonance imaging comprises magnetization/saturation transfer MRI. In some embodiments, the biomolecule is an hsp90 polypeptide. In some embodiments, the hsp90 polypeptide is a human hsp90 polypeptide (also referred to herein as an HSP90 polypeptide).

The presently disclosed subject matter also provides methods for selecting a therapy for a subject. In some embodiments, the methods comprise (a) providing to the subject a contrast enhancement agent comprising a small molecule that binds to a biomolecule present in a target tissue in the subject; (b) introducing the contrast enhancement agent into the target tissue; (c) scanning the target tissue using magnetic resonance imaging to determine if the target tissue overexpresses the biomolecule compared to a standard, whereby overexpression or lack of overexpression of the biomolecule in the target tissue provides a basis for selecting a therapy for the subject. In some embodiments, the biomolecule comprises an hsp90 polypeptide. In some embodiments, the target tissue comprises a plurality of cells that overexpress hsp90. In some embodiments, the target tissue is selected from the group including but not limited to an inflammatory lesion, a tumor, a pre-neoplastic lesion, a neoplastic cell, a pre-neoplastic cell, and a cancer cell. In some embodiments, the tumor is selected from the group including but not limited to a primary tumor, a metastasized tumor, and a carcinoma.

The presently disclosed subject matter also provides methods for delineating a boundary between a diseased cell and a non-diseased cell in a tissue in a subject. In some embodiments, the methods comprise (a) providing to the subject a contrast enhancement agent comprising a small molecule that binds to a biomolecule that is differentially expressed in the diseased cell and the non-diseased cell in the tissue; (b) introducing the contrast enhancement agent into the diseased cell and into the non-diseased cell in the tissue; and (c) scanning the tissue using magnetic resonance imaging whereby a boundary between a diseased cell and a non-diseased cell in the tissue in the subject is delineated. In some embodiments, the diseased cell is selected from the group including but not limited to a tumor cell, a pre-neoplastic cell, a neoplastic cell, and a cancer cell. In some embodiments, the boundary comprises a boundary between a tumor cell or a cancer cell and a normal cell in tissue surrounding the tumor cell or the cancer cell.

The presently disclosed subject matter also provides methods for assessing the degree to which a target tissue has been removed from a subject. In some embodiments, the methods comprise imaging a region in which the target tissue was present in the subject to assess for the degree to which cells that overexpress a biomolecule of interest have been removed from and/or remain in the target tissue. In some embodiments, the target tissue comprises a tumor or cancer cell that overexpresses hsp90.

The presently disclosed compositions and methods can be employed for imaging a target tissue in any subject for which the production of an image would be desirable. In some embodiments, the subject is a mammal. In some embodiments, the subject is a human.

Accordingly, it is an object of the presently disclosed subject matter to provide methods and compositions for non-invasive imaging of target tissues. This object is achieved in whole or in part by the presently disclosed subject matter.

An object of the presently disclosed subject matter having been stated above, other objects and advantages will become apparent to those of ordinary skill in the art after a study of the following description of the presently disclosed subject matter, drawings, and non-limiting examples.

### BRIEF DESCRIPTION OF THE DRAWINGS

FIG. 1 depicts concentration-dependent elution of hsp90 by novel small molecule based on one scaffold that binds hsp90.

FIG. 2 depicts co-crystallization of a small organic molecule with hsp90, confirming binding to the ATP site.

FIG. 3 is a plot depicting relative binding of novel small molecules selective for hsp90, as compared to the natural product 17-AAG (geldanomycin).

FIG. 4 is a bar graph depicting hsp90 probe accumulation in HT29 xenograft tumors in mice.

FIG. 5 depicts protein-mediated enhancement of small molecule imaging using frequency-selective saturation transfer MRI. The phantoms on the left demonstrate the increased sensitivity of small molecule imaging in the presence of protein. These phantoms more closely approximate the protein environment in vivo.

FIG. 6 depicts in vivo imaging of increased hsp90 expression in a mouse cerebral hemisphere (left image) following experimental spreading depression (SD). Right image is a control animal following SD but no small molecule administration. Immunhistochemical staining (not shown) demonstrated microglial reactivity that correlated with the area of increased signal intensity on MRI.

FIG. 7 depicts human brain tumor xenograft HSP90 expression, as fold increase over normal rat brain.

### DETAILED DESCRIPTION

### I. General Considerations

Proton MRI is based on the principle that the concentration and relaxation characteristics of protons in tissues and organs can influence the intensity and contrast characteristics of a magnetic resonance image. Contrast enhancement agents that are useful for proton MRI effect a change in the relaxation characteristics of protons, which can result in image enhancement and improved soft-tissue differentiation. Different classes of proton MRI agents include paramagnetic metal chelates and nitroxyl spin labeled compounds.

MRI is a diagnostic and research procedure that employs a large, high-strength magnet and radio frequency signals to produce images. The most abundant molecular species in biological tissues is water. It is the quantum mechanical “spin” of the water proton nuclei that ultimately gives rise to the signal in all imaging experiments. In an MRI experiment, the sample to be imaged is placed in a strong static magnetic field (typically on the order of 1-12 Tesla) and the spins are excited with a pulse of radio frequency (“RF”) radiation to produce a net magnetization in the sample. Various magnetic field gradients and other RF pulses then act on the spins to code spatial information into the recorded signals. The basic MRI experiment can be described, in one frame of reference, as follows: pre-RF pulse spins can be thought of as collectively aligned along the Z-axis of a Cartesian coordinate system; application of one or a sequence of RF pulses “tip” the spins into the X-Y plane, from which position they will spontaneously relax back to the Z-axis. The relaxation of the spins is recorded as a function of time. Using this basic experiment, MRI is able to generate structural information in three dimensions in a relatively short period of time.

MRI images are typically displayed on a gray scale with the color black representing the lowest measured intensity and white representing the highest measured intensity (I). This measured intensity is obtained by applying the formula I=C×M, where C is the concentration of spins (in an MRI experiment, this represents the water concentration) and M is a measure of the magnetization in the sample present at time of the measurement. Although variations in water concentration (C) can give rise to contrast in MRI images, it is the strong dependence of the rate of change in the magnetization (M) on local environment that is the major source of variation in image intensity in an MRI experiment.

Two characteristic relaxation times are implicated in magnetic relaxation, the basis for MRI. T₁ is defined as the longitudinal relaxation time, and is also known as the spin lattice relaxation time (1/T₁ is a rate constant, R₁, the spin-lattice relaxation rate constant). T₂ is known as the transverse relaxation time, or spin-spin relaxation mechanism, which is one of several contributions to T₂ (1/T₂ is also a rate constant, R₂, the spin-spin relaxation rate constant). T₁ and T₂ have inverse and reciprocal effects on image intensity, with image intensity increasing either by shortening the T₁ or lengthening the T₂.

In order to increase the signal-to-noise ratio (“SNR”), a typical MRI scan (RF and gradient pulse sequence and data acquisition) is repeated at a constant rate for a predetermined number of times and the data is subsequently averaged. The signal amplitude recorded for any given scan is proportional to the number of spins that have decayed back to equilibrium in the time period between successive scans. Thus, regions with rapidly relaxing spins (i.e. those regions comprising spins having short T₁ values) will recover all of their signal amplitude between successive scans. The measured intensities of the regions with long T₂ and short T₁ will reflect the spin density, which correlates with the region's water content. Regions with long T₁ values, as compared to the time between scans, will progressively lose signal (i.e. the signal linewidth will broaden and “flatten out”) until a steady state condition is reached. At the steady state condition, these regions will appear as darker regions in the final image. In extreme situations, the linewidth can be so large that the signal is indistinguishable from background noise.

Clinical MRI takes advantage of the fact that water relaxation characteristics vary from tissue to tissue, and this tissue-dependent relaxation effect provides image contrast, which in turn allows the identification of various distinct tissue types. Additionally, the MRI experiment can be set up so that regions of a sample with short T₁ values and/or long T₂ values are preferentially enhanced. Experiments so designed are known as T₁-weighted and T₂-weighted imaging protocols.

As has become known to those skilled in the art, however, coupling between the immobile, solid-like macromolecular protons and the mobile or “liquid” protons of water allows the spin state of the macromolecular protons to influence the spin state of the liquid protons through exchange processes. It is possible to saturate the spins of the immobile, solid-like macromolecular protons (“immobile macromolecular spins”) preferentially using an off-resonance radio frequency (RF) pulse. The immobile macromolecular spins have a much broader absorption lineshape than the spins of the liquid protons (“liquid spins”), making them as much as 106 times more sensitive to an appropriately placed off-resonance RF irradiation. This saturation of the immobile, solid-like macromolecular spins can be transferred to the liquid spins, depending upon the rate of exchange between the two spin populations, and hence is detectable with MRI. This process also is typically referred to as magnetization transfer (MT). See also Henkelman et al., 2001 and U.S. Pat. No. 5,050,609, the disclosure of each of which is incorporated herein by reference in its entirety.

Magnetization transfer is more than just a probe into the proton spin interactions within tissues as it also provides a mechanism that can be used to provide additional advantageous contrast in MR images. One application for use of the magnetization technique is in magnetic resonance angiography (MRA). In MRA specific imaging sequences are used to suppress the signal from static tissues while enhancing signal from blood by inflow or phase effects. The signal contrast between the blood and other tissue can be enhanced by using MT (which need not affect blood) to further suppress the background tissue signal. Better contrast between blood and tissue leads to better angiograms.

The phenomenon of magnetization transfer (MT) in MRI, alternatively referred to as exchange-based saturation transfer, was first described in biological tissues by Balaban and co-workers fifteen years ago (Wolff & Balaban, 1990). Over the past decade, clinical MRI protocols using magnetization transfer have been designed to partially saturate the contribution of macromolecular (lattice) protons for: (1) suppressing background signal in MR angiography (Ozsarlak et al., 2004); (2) improving paramagnetic contrast agent enhancement (Gaura et al., 2004); and (3) enhancing endogenous contrast from disease versus normal tissue (Mehta et al., 1996). Balaban and co-workers at the NIH were also the first to show proton chemical exchange between endogenous metabolites (e.g., ammonia, urea) and water in biological tissues (Guivel-Scharen et al., 1998).

The phenomenon of MT has since been developed for additional applications, including water exchange filter spectroscopy (WEX; Van Zijl et al., 2003), chemical exchange-dependent saturation transfer (CEST) imaging (Ward et al., 2000), and amide proton transfer (APT; Zhou et al., 2004) imaging. In each of these instances, the proton exchange process of MT is exploited to demonstrate magnetic coupling between water and mobile solutes containing exchangeable protons. In WEX and APT experiments, water-to-solute proton exchange is used to assess the exchange properties of amino, amine, SH, and OH groups (Zhou et al., 2003; Zhou et al., 2004). Peter van Zijl's group at Johns Hopkins has shown that spectral intensities of amide protons in APT can also serve as indicators for assessing cellular levels of mobile macromolecules and imaging pH (Zhou et al., 2003).

Balaban, van Zijl, and others including Dean Sherry at the University of Texas Southwestern (Dallas, Tex., United States of America) have shown that CEST imaging may be performed with agents that possess ideal proton exchange sites (Ward et al., 2000; Van Zijl et al., 2003; Zhang et al., 2003a). Numerous chemicals have been evaluated using this technique, with the most favorable exchange site found in amide protons (Ward et al., 2000). Van Zijl's group has exploited these inherent properties of amide protons to construct polymer complexes with large numbers of ideal exchange protons, allowing the detection of micromolar concentrations of macromolecules with the molar sensitivity of water (Goffeney et al., 2001; Snoussi et al., 2003). These dendrimer complexes could in theory be used as gene delivery systems that are imaged in vivo. This idea has been extended to paramagnetic lanthanide complexes by several groups that have shown that such complexes also have favorable exchange sites (Aime et al., 2002a; Aime et al., 2002b; Zhang et al., 2002; Aime et al., 2002c; Zhang et al., 2003a). Several paramagnetic CEST (or “PARACEST”) agents have now been reported that are capable of sensing indices such as pH (Aime et al., 2002a; Aime et al., 2002c; Zhang et al., 2002), lactate (Aime et al., 2002b), or glucose (Zhang et al., 2003b).

The present disclosure sets forth methods for magnetic resonance imaging of selective small molecules (i.e., low molecular weight small molecules) that bind specific cellular proteins, or that are taken up for specific cellular processes such as energy metabolism and protein and DNA synthesis. Also described are new approaches to designing ideal selective small molecule probes for in vivo magnetic resonance imaging. Together, the presently disclosed subject matter represents a strategy for in vivo imaging of selective small molecules without the need for contrast or radionuclide labeling.

Also disclosed herein is the targeting of non-exchangeable protons and amplification by virtue of direct transfer to bound proteins, the bound proteins in turn exchanging with mobile protons. Thus, the proteins become “amplifiers” of the signal. Another feature of the presently disclosed subject matter is the targeting of other nuclei for saturation transfer—“heteronuclear saturation transfer”.

Thus, aspects of the presently disclosed subject matter include but are not limited to 1) targeting saturation transfer pulses to protons typically viewed as nonexchangeable; (2) using endogenous proteins as amplifiers to generate contrast changes specific to the presence of small molecules; and 3) targeting nuclei other than protons (“heteronuclear saturation transfer”) contained within small molecules to compensate ultimately for magnetic field inhomogeneities and improve sensitivity/selectivity.

The use of MRI for molecular imaging strategies represents a major advance in clinical and basic in vivo imaging because of MRI's superior spatial resolution, capacity for concomitant anatomic and functional assessment, true multiplanar capability, and avoidance of radiation. Because MRI is already an accepted in vivo imaging modality, the development of this approach for in vivo imaging is believed to have widespread and immediate clinical application, since small molecule drugs already approved for use could in theory be used as imaging agents. The pharmacokinetic approaches to optimizing small molecule probes for MRI described herein also represent new strategies for designing novel molecular probes for imaging a multitude of disease states and their response to therapy.

### II. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used herein, including the claims, unless the context in which the term appears clearly indicates otherwise. Thus, for example, the phrases “a cell” and “the cell” can refer to one or more cells.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the term “angiogenesis” refers to the generation of new blood vessels into a tissue or organ. The term “angiogenesis” also specifically encompasses blood vessel growth in tumor tissues.

As used herein, the term “biological activity” refers to any observable effect flowing from an interaction between a biomolecule and a ligand.

As used herein, the term “detecting” refers to confirming the presence of a target entity by observing the occurrence of a detectable signal, such as a radiologic or spectroscopic signal that appears substantially exclusively, or is present at a meaningfully increased level, in the presence of the target entity.

As used herein, the term “inner transition elements” refers to those elements known as lanthanide (or rare earth) and actinide elements. Inner transition elements are also known as f-block transition elements.

As used herein, the term “labeling” refers to the attachment of a moiety, capable of detection by spectroscopic, radiologic, or other methods, to a probe molecule. Similarly, the moiety itself is referred to herein as a “label”, and such a probe molecule is referred to herein as “labeled”.

As used herein, the terms “metal chelator” and “chelator” are used interchangeably and refer to a molecule that forms a stable complex with a traceable metal atom under physiological conditions in that the metal remains bound to the conjugate in vivo.

As used herein, the term “modified” refers to an entity that is modified from its normally occurring state. An entity can be modified by removing discrete chemical units or by adding discrete chemical units. The term “modified” encompasses, but is not limited to detectable labels as well as those entities added as aids in purification.

As used herein, the term “modulate” refers an increase, decrease, or other alteration of any or all chemical and biological activities or properties of a biomolecule.

As used herein, the phrase “small molecule” refers to a compound, for example an organic compound, with a molecular weight of in some embodiments less than about 10,000 daltons, in some embodiments less than about 5,000 daltons, in some embodiments less than about 1,000 daltons, in some embodiments less than about 750 daltons, in some embodiments less than about 600 daltons, and in some embodiments less than about 500 daltons. A small molecule also can have a computed log octanol-water partition coefficient in some embodiments in the range of about −4 to about +14, and in some embodiments in the range of about −2 to about +7.5.

As used herein, the term “transition elements” means those elements found in columns IIIB, IVB, VB, VIIB, VIIIB IB, and IIB of the Periodic Table of Elements. Transition elements are also known as d-block elements.

### III. Methods for Imaging and Associated Technologies

The present disclosure describes in some embodiments methods for magnetic resonance imaging of selective small molecules (i.e., low molecular weight) that bind specific cellular proteins, or that are taken up for specific cellular processes such as energy metabolism, protein and DNA synthesis. These specific proteins and processes are often uniquely altered in various disease states. Therefore, these methods for imaging small molecules represent new approaches for in vivo magnetic resonance imaging of various disease processes and the response of disease processes to various therapies.

To further illustrate representative applications of the presently disclosed subject matter and its components, a few specific scenarios are described. Magnetic resonance imaging of selective small molecules in vivo would greatly aid the discernment of cancer from normal tissue, incorporating an imaging technique (MRI) which has the highest spatial resolution of any clinical imaging modality currently available, and which does not involve any radiation. In many cancers, such as prostate, breast, and brain (malignant gliomas), no imaging modalities currently exist which show sufficiently high specificity for routine diagnosis. A selective, sensitive, and specific molecular magnetic resonance imaging strategy for prostate cancer, for example, could replace the need for transrectal biopsy in the setting of elevated PSA levels. It could provide oncologists and surgeons an invaluable roadmap for assessing disease stage and surgical planning. It could provide a much more sensitive and rapid means for monitoring chemotherapy.

These advantages can be applied in theory to tumor imaging in almost any tissue type. With common metabolic, amino acid, and nucleotide substrates enriched with magnetic resonant nuclei other than H, metabolism, protein and DNA synthesis could be imaged for the first time in vivo with MR, without the need for radiation. The ability to measure these processes in vivo at high resolution could provide a highly sensitive approach for rapidly assessing treatment responses (e.g., cell division in the setting of malignancy), which was previously possible only in limited fashion with PET imaging in research settings due to the many challenges of this technology, including PET radiochemistry.

III.A. Methods for Non-Invasive Imaging

In some embodiments, the presently disclosed subject matter provides methods for non-invasively generating an image of a target tissue. In some embodiments, the methods comprise (a) providing a contrast enhancement agent comprising a small molecule that binds to a biomolecule present in the target tissue; (b) introducing the contrast enhancement agent into the target tissue; and (c) scanning the target tissue using magnetic resonance imaging, whereby an image of the target tissue is non-invasively generated.

In some embodiments, the target tissue is disposed within a subject. As used herein, the term “subject” refers to any organism for which diagnostic imaging and/or monitoring would be desirable. Thus, the term “subject” is desirably a human subject, although it is to be understood that the principles of the presently disclosed subject matter indicate that the presently disclosed subject matter is effective with respect to other species, including mammals, which are intended to be included in the term “subject”. Moreover, a mammal is understood to include any mammalian species for which diagnostic imaging and/or monitoring is desirable, particularly agricultural and domestic mammalian species. The methods of the presently disclosed subject matter are particularly useful in the diagnostic imaging and/or monitoring of warm-blooded vertebrates, e.g., mammals and birds.

More particularly, the presently disclosed subject matter can be used for diagnostic imaging and/or monitoring of a mammal such as a human. Also provided are methods for diagnostic imaging and/or monitoring in mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses (e.g., thoroughbreds and race horses). Also provided is the diagnostic imaging and/or monitoring of birds, including those kinds of birds that are endangered, or kept in zoos, as well as fowl, and more particularly domesticated fowl, e.g., poultry, such as turkeys, chickens, ducks, geese, guinea fowl, quail, pheasant, and the like, as they are also of economic importance to humans. Thus, provided is the diagnostic imaging and/or monitoring in livestock, including, but not limited to, domesticated swine (pigs and hogs), ruminants, poultry, and the like.

Various target tissues can be imaged using the compositions and methods disclosed herein. In some embodiments, the target tissue overexpresses a polypeptide relative to the surrounding tissues, facilitating the accumulation of the imaging agent in the target tissue as compared to the tissue surrounding the target tissue.

In some embodiments, the target tissue overexpresses hsp90 relative to the surrounding tissue. Heat Shock Protein 90 (hsp90 generally, or by convention, also referred to as HSP90 in the human form) is a molecular chaperone that guides the normal folding, intracellular disposition, and proteolytic turnover of many key regulators of cell growth and survival. Its function can be subverted during oncogenesis to facilitate malignant transformation possible, enhance rapid somatic evolution, and allow mutant proteins to retain or even gain function. Inhibition of hsp90 can slow these processes, and thus has potential therapeutic use (Whitesell & Lindquist, 2005). Accession numbers for human hsp90 (HSP90) amino acid sequences that are present in the GENBANK® database include NP_{—}001017963 (α1.1), NP_{—}005339 (α1.2), and AAF82792 (β).

Ansamycin antibiotics (e.g., herbimycin A (HA), geldanamycin (GM), and 17-allylaminogeldanamycin (17-AAG)) are thought to exert their anti-cancer effects by tight binding of the N-terminus pocket of hsp90, thereby destabilizing substrates that normally interact with hsp90 (Stebbins et al., 1997). This pocket is highly conserved and has weak homology to the ATP-binding site of DNA gyrase (Stebbins et al., 1997; Grenert et al., 1997). In vitro and in vivo studies have demonstrated that occupancy of this N-terminal pocket by ansamycins and other hsp90 inhibitors alters hsp90 function and inhibits protein folding. At high concentrations, ansamycins and other hsp90 inhibitors have been shown to prevent binding of protein substrates to hsp90 (Scheibel et al., 1999; Schulte et al., 1995; Whitesell et al., 1994). Ansamycins have also been demonstrated to inhibit the ATP-dependent release of chaperone-associated protein substrates (Schneider et al., 1996; Sepp-Lorenzino et al., 1995). In either event, the substrates are degraded by a ubiquitin-dependent process in the proteasome (Schneider et al., 1996; Sepp-Lorenzino et al., 1995; Whitesell et al., 1994). Hsp90 substrate destabilization occurs in tumor and non-transformed cells alike and has been shown to be especially effective on a subset of signaling regulators including, but not limited to Raf (Schulte et al., 1997; Schulte et al., 1995), nuclear steroid receptors (Segnitz & Gehring, 1997; Smith et al., 1995), v-Src (Whitesell et al., 1994), and certain transmembrane tyrosine kinases (Sepp-Lorenzino et al., 1995) such as EGF receptor (EGFR) and HER2/Neu (Hartmann et al., 1997; Miller et al., 1994; Mimnaugh et al., 1996; Schnur et al., 1995), CDK4, and mutant p53 (Erlichman et al., 2001). The ansamycin-induced loss of these proteins leads to the selective disruption of certain regulatory pathways and results in growth arrest at specific phases of the cell cycle (Muise-Heimericks et al., 1998), apoptosis, and/or differentiation of cells so treated (Vasilevskaya et al., 1999). Molecules that bind to hsp90 thus hold great promise for imaging and/or monitoring of many types of cancers and proliferative disorders.

In some embodiments of the presently disclosed subject matter, the target tissue is selected from the group including but not limited to an inflammatory lesion, a tumor, a pre-neoplastic lesion, a neoplastic cell, a pre-neoplastic cell, and a cancer cell (e.g., a primary tumor, a metastasized tumor, or a carcinoma). More particularly, the presently disclosed subject matter provides in some embodiments methods for imaging a tumor, cancer, or carcinoma that is characterized by upregulated expression of hsp90. Examples of such tumors, cancers, or carcinomas include, but are not limited to certain prostate tumors, breast tumors, uterine tumors, melanomas, gliomas, bladder carcinomas, laryngeal carcinomas, salivary gland tumors, and leukemias.

III.B. Methods for Monitoring a Response to a Therapy

In some embodiments, the presently disclosed subject matter provides methods for monitoring a response to a therapy in a target tissue in a subject. In some embodiments, the methods comprise comparing a plurality of MRI images of a target tissue in the subject, wherein (a) a first subset of the plurality of MRI images are images of the target tissue generated prior to administering the therapy to the subject; and (b) a second subset of the plurality of MRI images are images of the target tissue generated at a time subsequent to administering the therapy to the subject and at which a response to the therapy is expected; and further wherein the plurality of MRI images are generated by a method comprising (i) providing a contrast enhancement agent comprising a small molecule that binds to a biomolecule present in the target tissue; (ii) introducing the contrast enhancement agent into the target tissue; and (iii) scanning the target tissue using magnetic resonance imaging, whereby a visible image of the target tissue is non-invasively generated.

III.C. Methods for Selecting a Therapy for a Subject

The presently disclosed subject matter also provides methods for selecting a therapy for a subject. In some embodiments, the methods comprise (a) providing to the subject a contrast enhancement agent comprising a small molecule that binds to a biomolecule present in a target tissue in the subject; (b) introducing the contrast enhancement agent into the target tissue; (c) scanning the target tissue using magnetic resonance imaging to determine if the target tissue overexpresses the biomolecule compared to a standard, whereby overexpression or lack of overexpression of the biomolecule in the target tissue provides a basis for selecting a therapy for the subject.

In some embodiments, the therapy comprises surgical resection of a diseased tissue. As such, the presently disclosed subject matter also provides methods for delineating a boundary between a diseased cell and a non-diseased cell in a tissue in a subject. In some embodiments, the methods comprise (a) providing to the subject a contrast enhancement agent comprising a small molecule that binds to a biomolecule that is differentially expressed in the diseased cell and the non-diseased cell in the tissue; (b) introducing the contrast enhancement agent into the diseased cell and into the non-diseased cell in the tissue; (c) scanning the tissue using magnetic resonance imaging whereby a boundary between a diseased cell and a non-diseased cell in the tissue in the subject is delineated.

### IV. Compositions

IV.A. Generally

In some embodiments, the presently disclosed subject matter provides a novel approach to designing selective small molecule probes for in vivo magnetic resonance imaging. In some embodiments, the small molecule magnetic resonance imaging strategies disclosed herein employ a relative high concentration (100 nM or greater) of small molecule probes in localized compartments (e.g., diseased cells) for detection. The presently disclosed methods thus in some embodiments employ selective small molecules that sequester in these “compartments” while not accumulating in normal cells and tissues, not only avoiding unwanted toxicities but also achieving the required high target-to-background ratios optimal for imaging contrast.

These unique parameters can be met through the rational design of super-selective low affinity small molecules that target proteins that are locally high in concentration. Where target protein concentrations are locally high (e.g., intracellular compartment), low affinity small molecules become trapped through rebinding phenomenon; where target protein concentrations are low (e.g., normal cells and tissues), low affinity small molecules demonstrate no significant accumulation since they are only weak binders.

Low molecular weight small molecules (e.g., less than about 500 Daltons) represent an important class of organic chemicals with proven utility for exploring cell function at the molecular level, and currently comprise the majority of marketed drugs and molecular imaging probes. Imaging of small molecule probes is usually accomplished by radionuclide labeling for PET or SPECT, Radionuclide techniques, although sensitive, suffer from low spatial resolution, lack of concomitant anatomical and functional information, and altered biological activity following radiolabeling, which itself is expensive and often difficult to achieve. The presently disclosed subject matter provides in some embodiments a method for in vivo imaging of small molecules using frequency-selective magnetization transfer MRI, which targets saturation pulses to the unique chemical shifts of a given small molecule based on initial magnetic resonance (MR) proton spectroscopic characterization. This method, therefore, represents an approach for in vivo imaging selective small molecules without the need for contrast or radionuclide labeling.

In some embodiments, the presently disclosed subject matter also relates to low molecular weight imaging probes with resonant nuclei (e.g., ¹³C, ²³Na, ¹⁹F, ¹⁴N, ³¹P) which in some embodiments occur in nature, but at much lower frequencies, and can either be endogenous to, or actively substituted into, small molecules, to enhance sensitivity and selectivity of saturation transfer from small molecule species in vivo. This aspect of the presently disclosed subject matter allows broader bandwidth saturation pulses, compensating for magnetic field inhomogeneities, and increases specificity against background endogenous proton spectra. This aspect of the presently disclosed subject matter also allows for selective MRI in vivo of small molecules such as metabolic substrates for imaging metabolism, amino acids for imaging protein synthesis, and nucleotide bases for imaging DNA synthesis, processes which have only been imaged in vivo to date in limited circumstances using PET imaging. Nuclear substitution is not considered essential to imaging novel small molecule probes, especially if a given low molecular weight species possesses useful proton spectra sufficiently outside the range of free water and endogenous tissue proton spectra. Nuclear substitution, however, is essential for imaging metabolic substrates (e.g., glucose), amino acids (protein synthesis) and nucleotide bases (DNA and RNA synthesis), since these small molecule substrates are ubiquitous in vivo.

A second aspect of the presently disclosure subject matter describes the complimentary rational design of novel, super-selective small molecule imaging probes which are optimally designed for frequency-selective magnetization transfer MRI. A long-standing tenet of drug and molecular imaging probe development is that candidate molecules must exhibit high affinity for their intended target. In contradistinction, disclosed herein is the design and use of low affinity small molecules that nonetheless have high avidity for their targets in the setting of locally high target concentration through rebinding phenomenon. Locally high target concentrations are seen with various small molecule-binding species such as chaperone proteins and various metabolic enzymes that are often dramatically upregulated in cancer and a variety of other disease states. The design of low affinity, high avidity low molecular weight probes and drugs represents a novel approach to achieving probe or drug accumulation in target tissue (e.g., cancer cells) while avoiding background distribution in normal cells and tissue. This approach has application not only for new molecular imaging methods but also novel therapeutic strategies.

IV.B. Routes of Administration

The presently disclosed imaging compositions can be administered to a subject in any form and/or by any route of administration. In some embodiments, the imaging agent is selected from the group including but not limited to an oral formulation, a peroral formulation, a buccal formulation, an enteral formulation, a pulmonary formulation, a rectal formulation, a vaginal formulation, a nasal formulation, a lingual formulation, a sublingual formulation, an intravenous formulation, an intraarterial formulation, an intracardial formulation, an intramuscular formulation, an intraperitoneal formulation, an intratumoral formulation, an intracranial formulation, an intracutaneous formulation, a subcutaneous formulation, an aerosolized formulation, an ocular formulation, an implantable formulation, a depot injection formulation, and combinations thereof. In some embodiments, the route of administration is selected from the group including but not limited to oral, peroral, buccal, enteral, pulmonary, rectal, vaginal, nasal, lingual, sublingual, intravenous, intraarterial, intracardial, intramuscular, intraperitoneal, intracranial, intracutaneous, intratumoral, subcutaneous, ocular, via an implant, and via a depot injection. Where applicable, continuous infusion can enhance accumulation of an imaging composition at a target site (see, e.g., U.S. Pat. No. 6,180,082). In some embodiments, the imaging agents of the presently disclosed subject matter are administered intravenously, and in some embodiments of the presently disclosed subject matter the imaging agents of the presently disclosed subject matter are administered intratumorally.

IV.C. Formulations

An imaging composition as described herein comprises in some embodiments a composition that includes a pharmaceutically acceptable carrier. Suitable formulations include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents.

The formulations can be presented in unit-dose or multi-dose containers, for example sealed ampoules and vials, and can be stored in a frozen or freeze-dried (lyophilized) condition requiring only the addition of sterile liquid carrier immediately prior to use.

The imaging agents can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt).

The imaging agents can also be formulated in rectal compositions (e.g., suppositories or retention enemas containing conventional suppository bases such as cocoa butter or other glycerides), creams or lotions.

In some embodiments, the presently disclosed subject matter employs an imaging composition that is pharmaceutically acceptable for use in humans. One of ordinary skill in the art understands the nature of those components that can be present in an imaging composition that is pharmaceutically acceptable for use in humans and also what components should be excluded from an imaging composition that is pharmaceutically acceptable for use in humans.

IV.D. Doses

The term “effective amount” is used herein to refer to an amount of an imaging composition (e.g., a composition comprising an imaging agent) sufficient to produce a visible image. Actual dosage levels of an imaging composition of the presently disclosed subject matter can be varied so as to administer an amount of the imaging agent that is effective to achieve the desired imaging for a particular subject and/or application. The selected dosage level can depend upon a variety of factors including the affinity and/or avidity of the imaging agent for the biomolecule to which it is intended to bind, the formulation, the route of administration, and the abundances of the biomolecule in the target tissue and the surrounding tissue in the subject being imaged. In some embodiments, a minimal dose is administered, and dose is escalated in the absence of dose-limiting toxicity to a minimally effective amount. Determination and adjustment of an effective dose, as well as evaluation of when and how to make such adjustments, are known to those of ordinary skill in the art of medicine.

For administration of an imaging composition as disclosed herein, conventional methods of extrapolating human dosage based on doses administered to a murine animal model can be carried out using techniques known to one of ordinary skill in the art. Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich et al., 1966. Briefly, to express a mg/kg dose in any given species as the equivalent mg/m² dose, multiply the dose by the appropriate km factor. In an adult human, 100 mg/kg is equivalent to 100 mg/kg×37 kg/m²=3700 mg/m².

### EXAMPLES

The following Examples provide illustrative embodiments. Certain aspects of the following Examples are disclosed in terms of techniques and procedures found or contemplated by the present inventors to work well in the practice of the embodiments. In light of the present disclosure and the general level of skill in the art, those of skill will appreciate that the following Examples are intended to be exemplary only and that numerous changes, modifications, and alterations can be employed without departing from the scope of the presently claimed subject matter.

### Example 1

### NMR Spectroscopic Characterization of Chloroquine

The method of frequency-selective saturation transfer MR described hereinabove was employed to image chloroquine in vitro and its sequestration in the mouse retina in vivo. Initial spectroscopic characterization of 100 mM chloroquine was performed on a GE 4T using a single voxel PRESS sequence with TRITE=2000/30 ins. NMR imaging of chloroquine demonstrates several peaks. The cluster of peaks at 1.15 ppm was targeted for off-resonance saturation transfer.

### Example 2

### Magnetic Coupling of Chloroquine to Water Protons In Vitro

Solutions for six 50 cc model phantoms were prepared as follows: (1) 100 mM chloroquine in water, pH 7.4; (2) 100 mM NaPO₄ in water, pH 7.4; (3-6) 20% (w/w) bovine serum albumin (BSA), cross-linked with 1.25 cc 25% glutaraldehyde, with 10 μM, 1 mM, 50 mM, and 100 mM chloroquine, respectively. Magnetization transfer imaging experiments were performed on a GE 4T using a spin echo pulse sequence with TR/TE=1000/10 ms, preceded by a series of 16 frequency selective saturation pulses centered at a spectral peak unique to chloroquine. Two standard images were acquired: one with frequency-selective saturation set on the 1.15 ppm peak of chloroquine, and a control image with saturation set symmetrically on the other side of water to subtract the MT effect from macromolecules. Image subtraction yielded an image of chloroquine magnetically coupled to water protons.

Imaging experiments showed a reduction in the free water signal following saturation pulses targeted both to chloroquine at 1.15 ppm, and to a symmetric off-resonance frequency on the opposite side of water. Subtraction of the former from the latter images yielded a difference in MT reduction in the free water signal that scaled with chloroquine concentration. Chloroquine coupling to free water was only seen in the presence of macromolecules (BSA), and was demonstrable to concentrations <1 mM using only a single targeted saturation pulse train of 256 ms.

### Example 3

### NMR Imaging of Chloroquine in the Mouse Retina In Vivo

Utilizing the MR pulse sequence developed for selectively imaging chloroquine in phantoms, the accumulation of chloroquine was imaged in vivo in mouse retina. Chloroquine (Sigma Chemical Co., St. Louis, Mo., United States of America) was dissolved in H₂O and titrated to pH 7.2. The drug was administered via intraperitoneal injection for 2 or 4 days at 100 mg/Kg to C57 mice using an approximate volume of ice per dose. No signal intensity was seen in the absence of chloroquine administration.

### Example 4

### Determination of Affinity for hsp90

Affinity of test compounds for hsp90 is determined as per the procedure set forth in EXAMPLE 49 of United States Patent Application Publication 20060211737 (corresponding to U.S. patent application Ser. No. 11/363,449), which is hereby incorporated by reference in its entirety. Briefly, protein mixtures obtained from a variety of organ tissues (for example: spleen, liver and lung) are reversibly bound to a purine affinity column to capture purine-binding proteins, especially hsp90. The purine affinity column is washed several times, and then eluted with 20 μM, 100 μM, and 500 μM of test compound. Candidate compounds elute hsp90 in a dose-dependent manner vs. a control elution using dimethylsulfoxide.

The elution profile of candidate compounds is determined by 1-dimensional SDS polyacrylamide gel electrophoresis. Gels are stained with a fluorescent stain such as sypro ruby (a highly sensitive fluorescent protein stain that can readily detect less than 1 fmol of total protein, i.e., less than 0.04 ng for a 40 kDa protein) or silver nitrate. The gels are imaged using a standard flat bed gel imager and the amount of protein estimated by densitometry. The percent of hsp90 protein eluted from the column at each concentration is determined and IC₅₀ values are calculated from these estimates. The identity of a band containing hsp90 is determined by protein sequencing using mass spectroscopy.

### Discussion of the Examples 1-4

As disclosed herein, magnetic coupling between chloroquine and water was demonstrated in a concentration-dependent fashion using frequency-selective saturation transfer pulses targeted to the dominant chloroquine peak after initial spectroscopic characterization. Indirect imaging of chloroquine via MT depended on the presence of macromolecules (e.g., BSA). The selective imaging of chloroquine in a model system in vitro and in vivo represents a prototype demonstration of exogenous small molecule MR imaging, without the need for chemical alteration through contrast or radionuclide labeling.

Low molecular weight small molecules represent an important class of organic chemicals with proven value for exploring cell function at the molecular level, and currently comprise the majority of marketed drugs and molecular imaging probes. Imaging of small molecule probes is usually accomplished by radionuclide labeling for PET or SPECT. Radionuclide techniques, although sensitive, suffer from low spatial resolution, lack of concomitant anatomical and functional information, and altered biological activity following radiolabeling, which itself is expensive and often difficult to achieve.

The presently disclosed subject matter sets forth inter alia new methods for magnetic resonance imaging of selective small molecules (i.e., low molecular weight molecules) that bind to specific cellular proteins and/or that are taken up for specific cellular processes such as energy metabolism, protein and DNA synthesis. Also described are novel approaches to designing ideal selective small molecule probes for in vivo magnetic resonance imaging. The presently disclosed subject matter thus provides strategies for in vivo imaging of selective small molecules without the need for contrast or radionuclide labeling.

The use of MR for molecular imaging strategies represents a major advance in clinical and basic in vivo imaging because of MRI's superior spatial resolution, capacity for concomitant anatomic and functional assessment, true multiplanar capability, and avoidance of radiation. Because MRI is already an accepted in vivo imaging modality, the development of these approaches for in vivo imaging is expected to have widespread and immediate clinical application, since small molecule drugs already approved for use could be used as imaging agents. The unique pharmacokinetic approaches to optimizing small molecule probes for MR imaging disclosed herein also represent new strategies for designing novel molecular probes for imaging a multitude of disease states and their response to therapy.

Disclosed herein is the application of saturation transfer phenomenon for in vivo imaging in some embodiments of unmodified small molecule drugs selective for known protein targets. Prior reports have only described saturation transfer imaging of a limited number of endogenous metabolites, polymer complexes, or paramagnetic lanthanide complexes.

Also disclosed herein are methods in which magnetic coupling between water protons (from which MR signals for imaging are generated) and small molecule drugs can be attained by targeting non-exchangable protons as well as exchangeable protons, greatly facilitating the ability to image potentially any small molecule drug as a novel MRI contrast agent. Sensitivity can be attained by amplification via endogenous proteins that bind these small molecules. In other words, instead of using lanthanide chelates or dendrimer complexes for amplification, or exchangeable protons on small molecules that might not be present or accessible, endogenous macromolecular amplification, which necessarily occurs when a selective small molecule drug binds endogenous protein, is co-opted. The protein targets, and other intracellular proteins which can bind small molecules non-specifically, become macromolecular amplifiers allowing for detection of micromolar concentrations of small molecules with the molar sensitivity of water.

Additionally described herein are methods of saturation transfer of other resonant nuclei (e.g., ¹³C, ²³Na, ¹⁹F, ¹⁵N, ³¹P) which can either be endogenous to, or actively substituted into, small molecules to enhance sensitivity and selectivity for imaging small molecule species in vivo. This aspect of the presently disclosed subject matter allows for broader bandwidth saturation pulses, compensating for magnetic field inhomogeneities, and increases specificity against background endogenous proton spectra. This aspect of the presently disclosed subject matter also allows for selective MRI in vivo of small molecules such as metabolic substrates for imaging metabolism, amino acids for imaging protein synthesis, and nucleotide bases for imaging DNA synthesis, processes which to date have only been imaged in vivo date in limited circumstances using PET imaging. This method of saturation transfer imaging involves first saturating resonant nuclei other than H¹, allowing transfer phenomenon to occur via dipolar interactions with covalently-linked protons, then generating contrast between these linked protons and water via magnetization transfer.

Also disclosed herein is the rationalized design of selective small molecule imaging probes with high avidity but lower affinities for high copy targets intentionally achieved to attain high target-to-background ratios and low toxicity. A long-standing tenet of drug and imaging probe development holds that achieving high affinity for molecular targets is paramount. However, many useful molecular targets that are highly expressed in diseased cells are also expressed at some lower, basal level in normal cells. When high affinity drugs or imaging probes are administered in vivo, they often distribute in both normal cells and tissue as well diseased cells, producing unwanted binding and toxicity and reducing target to background ratio.

Low affinity molecules, on the other hand, are designed to not distribute and accumulate in normal cells where protein targets are low in copy, but to sequester in target cells where targets are high in copy through rebinding phenomenon. Low affinity small molecules which accumulate in compartments (e.g., intracellular) through rebinding phenomenon can be said to have high avidity for their target in the appropriate setting—that is, high localized concentrations of protein target. Although applicants do not wish to be bound by any particular theory of operations, it is possible that continuous rebinding of probe-to-target occurs following dissociation of the target-probe complex and competes favorably with diffusion away from the compartment where binding occurs. In other words, low affinity small molecules in the setting of compartmentalized high protein target concentration can have a greater probability of rebinding to unoccupied targets than diffusing out of the compartment. Small molecules that compartmentalize only in the setting of high target concentration have high avidity for their target despite low affinity, produce high target to background ratios and less toxicity. These characteristics are ideal for molecular imaging probes.

To applicants' knowledge, there are no reports of the intentional design of low affinity, high avidity small molecule drugs or probes. The phenomenon of probe-target rebinding, however, has received limited treatment. Perhaps the most detailed theoretical and empirical treatment is found in Frost & Wagner, 1984, which addressed the pharmacokinetic observation that in regions of the brain where opiate receptor densities are high, ligands (labeled drug and PET imaging probes) clear from the brain more slowly that in regions of the brain where the receptors are absent or in lower copy. Drawing on their theoretical treatment and empirical studies, one can predict the accumulation of molecules in compartments with high target copy despite possessing low affinity for their intended target.

Thus, it is possible that high avidity through rebinding phenomenon might be the basis of accumulation disclosed herein of the small molecule drug chloroquine in the retina, despite the drug's low affinity for one of its two known protein targets, aldehyde dehydrogenase 1, which is expressed in high copy in this tissue (see EXAMPLES 1-4).

### Example 5

### Small Molecule Development

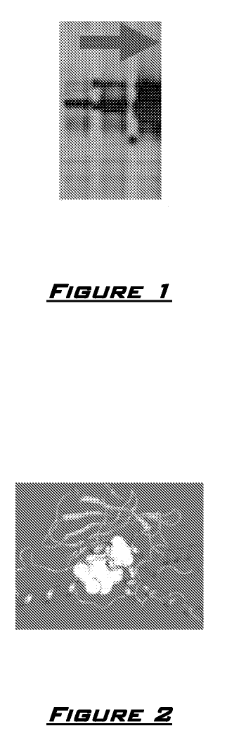

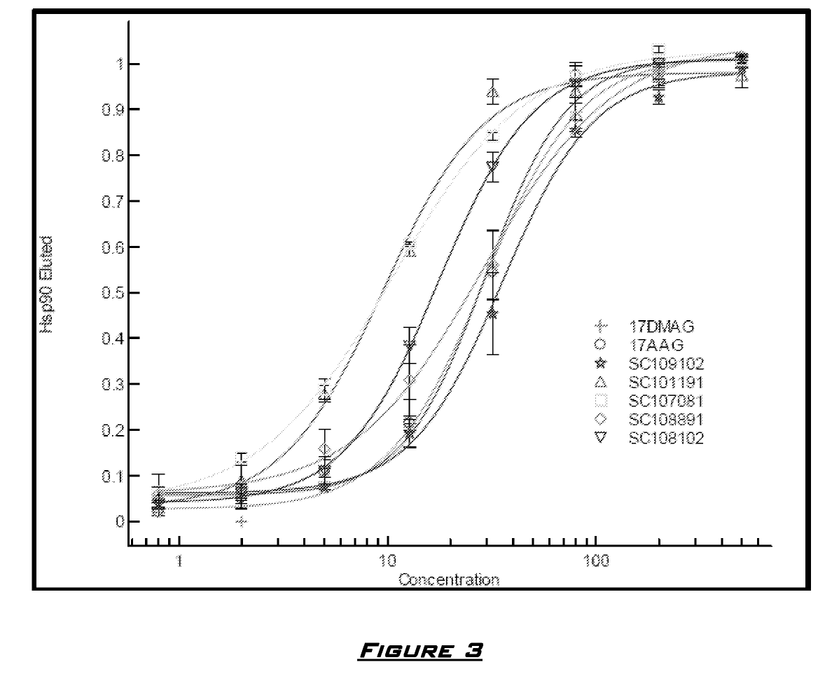

Small molecules selective for Hsp90 have been developed. A library of small molecule candidates is screened against proteins immobilized by virtue of their purine-binding ability (e.g., ATP-binding proteins). The isolation of this purine utilizing subproteome, or “purinome”, has been described previously (Graves et al., 2002). FIG. 1 demonstrates an example of small molecule that selectively elutes Hsp90 in concentration-dependent fashion. FIG. 2 shows the 3-D crystal structure of a selective small molecule bound to Hsp90, confirming that selective binding occurs within the ATP-binding pocket of hsp90. Small organics that successfully elute Hsp90 are modified through iterative medicinal chemistry methods to optimize serum half-life, biodistribution, toxicity profile, and lipophilicity, while maintaining Hsp90 selectivity. The relative affinities of a panel of novel small molecules for Hsp90 are seen in FIG. 3, and are compared to 17-AAG, or geldanomycin, a natural product that binds Hsp90 and is currently being tested in human trials as an anti-neoplastic agent.

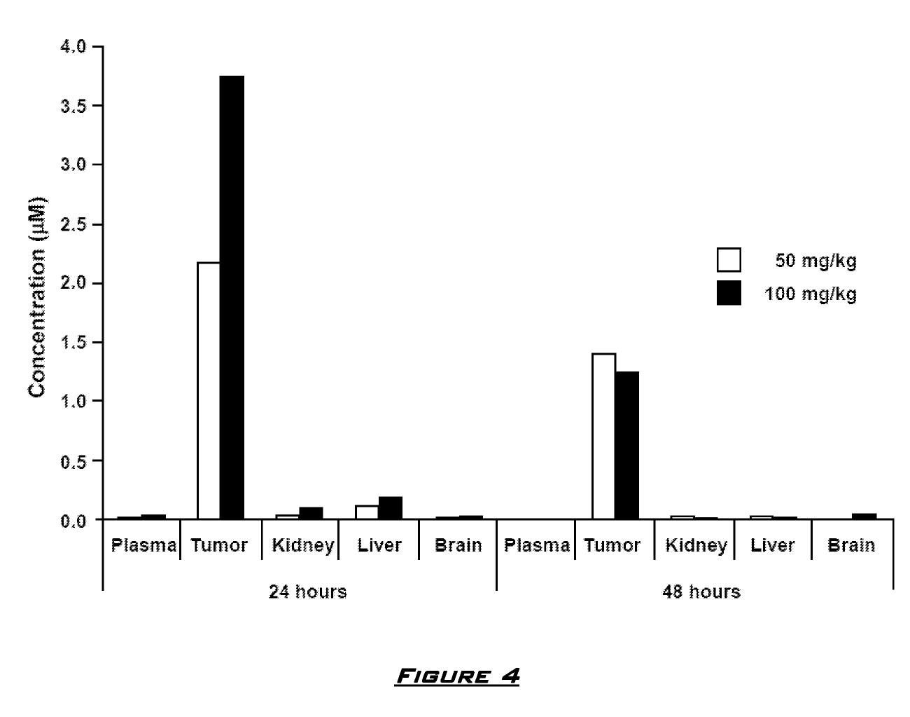

For saturation transfer imaging methods to be feasible, probes must accumulate in cellular compartments at high concentration (micro to millimolar) in the setting of high target expression. Micromolar accumulation of a candidate small molecule in xenograft tumors after only a single dose administration is depicted in FIG. 4.

### Example 6

### Small Molecule Magnetic Resonance Imaging

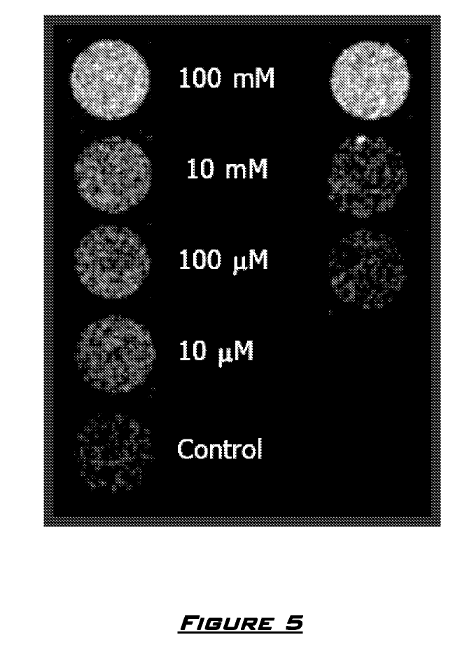

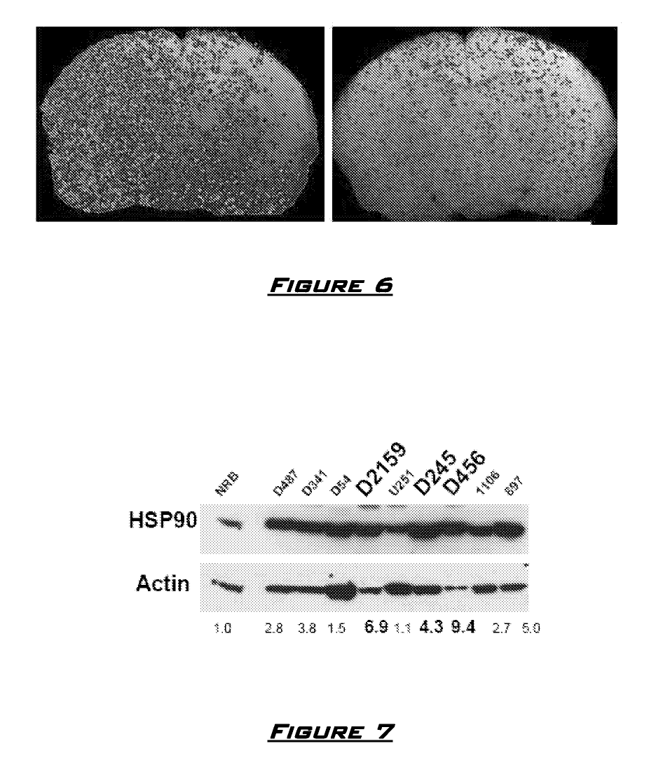

Prior studies have shown magnetic coupling between water and small molecules such as ethanol (Eshalai, 2003) and lactate (Swanson, 1998) mediated through protein. In preliminary phantom experiments, increased sensitivity of several orders of magnitude in the detection of small molecules selective for Hsp90 in the presence of cross-linked protein was demonstrated (see FIG. 4). These phantoms more closely approximated imaging characteristics of biological tissue. Saturation transfer imaging experiments used a spin echo pulse sequence with TR/TE=1000/10 ms, proceeded by a series of 16 frequency selective saturation pulses centered on the 1.15 ppm peak of a selective Hsp90 inhibitor, and a control peak symmetrically on the other opposite side of water (4.7 ppm) to subtract the contribution from macromolecules. Image subtraction yields an image of the small molecule magnetically coupled to water protons that scales with small molecule concentration (FIG. 5). The pulse sequence developed in phantoms has also been applied in vivo following intraperitoneal administration of the small molecule selective for hsp90 in an experimental model of hemicortical hsp90 upregulation (FIG. 6). No differential detection of the small molecule was seen in the absence of hsp90 expression or probe administration.

### Discussion of Examples 5 and 6

An aim of the presently disclosed subject matter is to develop novel molecular magnetic resonance imaging strategies for noninvasive diagnostic visualization of cancer and precancerous lesions currently that are undetectable using conventional imaging methods. The presently disclosed subject matter combines novel chemoproteomic and magnetic resonance imaging methods for detecting orally bioavailable small organic molecules that selectively bind to heat shock 90 (hsp90) proteins, an established purine-binding protein biomarker highly expressed in, for example, prostate, breast, and brain cancer. A series of novel small organic molecule candidates have been established that bind to hsp90 selectively and that demonstrate favorable pharmacokinetic properties. Simultaneously, new strategies for in vivo magnetic resonance imaging of these small molecules based on the phenomenon of chemically selective saturation transfer have been developed. The disclosed molecular magnetic resonance imaging approach are tested in animal models of breast, prostate, and brain cancer, among others, followed by studies in humans based on the 1) success of the initial feasibility experiments; and 2) the advanced preclinical stage of the same hsp90 inhibitors as anticancer agents. The molecular MR approaches disclosed herein for imaging the bioaccumulation of hsp90 inhibitors in tumors is expected to significantly aid diagnosis, treatment planning, and therapy monitoring of poorly detectable malignancies, and is intended to have widespread clinical application since MRI is already an accepted imaging modality

Clinical imaging techniques are currently insufficient for adequately characterizing or localizing cancerous lesions in certain organs including, but not limited to prostate, breast, and brain. In each case, direct tissue analysis is required for definitive diagnosis, yet biopsies are often diagnostically inadequate since tumors and organs are not sampled in their entireties. In prostate cancer, transrectal biopsy is commonly nonspecific since it is performed almost blindly under limited ultrasound guidance. As such, prostate biopsies demonstrate poor diagnostic yield, with up to 25% of repeated biopsies revealing cancerous lesions after initial specimens are declared negative (Donahue et al., 2002). Poor localization of true tumor margins, especially in prostate cancer and gliomas, hinders surgeons' ability for complete resection and potential cure.

The development of a high resolution noninvasive imaging technique that combines detailed anatomy with molecular specificity for malignancy would therefore represent a major breakthrough in noninvasive cancer assessment. In prostate cancer, for example, such a technique could guide, if not replace, transrectal biopsy by identifying tumor cells in a heterogeneous but otherwise amorphous gland. A sensitive, high resolution imaging technique would also facilitate pre-surgical planning by revealing detailed tumor localization and extension (e.g., staging), as well as provide a technique for monitoring tumor response to less invasive treatment options such as cryotherapy, radiation, and chemotherapy.

Successful in vivo molecular imaging strategies require the integrated solution of several research pathways: identification of selective cellular targets, discovery of high affinity biocompatible probes which bind these targets, and development of technology for visualizing these targets. By focusing on the purine-binding subproteome for biomarker identification, it is possible to simultaneously solve the problems of cellular target and selective probe discovery since each purine-binding protein, by virtue of its structure, is potentially amenable to highly selective binding by small organic molecules. Many purine-binding proteins, such as heat shock proteins, metabolic enzymes, carboxylases, and dehydrogenases, are highly upregulated in many malignancies, and often selectively in different states of tumor development. Small molecule probes that selectively bind ATP-binding biomarkers in turn can be optimized to possess favorable pharmacokinetic properties and low toxicities through iterative medicinal chemistry.

The experiments disclosed herein are designed to test a panel of small molecule probes which selectively bind heat shock 90 (hsp90), a purine-binding protein already established as a biomarker for prostate, breast, brain, and other malignancies (Strik et al., 2000; Zheng et al., 2001; Sauer et al., 2002; Zellweger et al., 2005).

The presently disclosed strategies for imaging hsp90 serve as a prototype for designing and visualizing small molecule probes selective for other purine-binding proteins upregulated in various cancerous and precancerous lesions. A research group in Duke's Department of Pathology, for example, has recently discovered that another heat shock protein, GRP78, a member of the hsp70 family, might be a highly selective biomarker for distinguishing more malignant forms of prostate cancer (Misra et al., 2002). By applying the presently disclosed molecular MR imaging approach to novel biomarkers such as grp78, it should be possible to distinguish prostate malignancies requiring aggressive therapy from those to be followed without treatment using noninvasive imaging.

This present disclosure describes the application of a successful drug development strategy to molecular imaging probe development in order to achieve high imaging selectivity and favorable pharmacokinetics. Disclosed is the use of low molecular weight (<500 Daltons) drug-like small molecules, a class of molecules that currently comprises the majority of marketed pharmaceuticals and molecular imaging probes. Imaging of small molecule probes is usually accomplished by radionuclide labeling for PET or SPECT. Radionuclide techniques, although sensitive, suffer from low spatial resolution, lack of concomitant anatomical and functional information, and altered biological activity following radiolabeling, which itself is expensive and often difficult to achieve. The present disclosure describes methods for in vivo imaging of small molecules using frequency-selective saturation transfer MRI, without the need for contrast or radionuclide labeling.

The use of MR for molecular imaging would represent a major advance in clinical and basic in vivo imaging because of MRI's superior spatial resolution, capacity for concomitant anatomic and functional assessment, true multiplanar capability, and avoidance of radiation. Because MRI is already an accepted in vivo imaging modality, and frequency-selective magnetization transfer has the potential to generate specific small molecule contrast without ad hoc chemical labeling of imaging probes, this approach to in vivo imaging could have widespread and immediate clinical application.

Therefore, in some embodiments the presently disclosed subject matter relates to the development of frequency-selective saturation transfer MR imaging sequences for imaging small molecule probes selective for hsp90. While applicants do not wish to be bound by any particular theory of operation, it is possible that magnetic coupling between small molecule probes and water protons allows the use of targeted saturation transfer techniques to generate contrast specific to small molecules in vivo, with sensitivities at least three orders of magnitude greater than conventional MR contrast mechanisms.

Recent studies have demonstrated the feasibility of utilizing saturation transfer to generate MR contrast specific to endogenous metabolites, dendrimer and lanthanide complexes (Guivel-Scharen et al., 1998; Zhang et al., 2003b; Goffeney et al., 2001). Additional work has shown magnetic coupling in vitro and in vivo between amino acids, sugars, and other biological molecules (Ward et al., 2000). Saturation transfer can be optimized by targeting certain proton exchange sites such as amide and hydroxyl protons (Newby & Greenbaum, 2002). With non-exchangeable protons, such as those in methyl groups, saturation transfer between small molecules and water is achieved in the presence of protein (Eshalai, 2003; Swanson, 1998). In preliminary phantom experiments disclosed herein, micromolar sensitivity for detection of small molecules in the presence of cross-linked protein has been demonstrated. These phantoms more closely approximate imaging characteristics of biological tissue as compared to aqueous or uncross-linked protein solutions. Experiments in this area further optimize the ability to detect these small molecules by investigating the role of various MR parameters such as saturation energy and angle, and characteristics of slice-selective pulse sequences that influence small molecule MR contrast.

In some embodiments, the presently disclosed subject matter relates to imaging small molecule probes selective for hsp90 in mouse models of prostate, breast, and brain cancer using saturation transfer MR sequences as set forth hereinabove. While applicants do not wish to be bound by any particular theory of operation, it is possible that since preliminary experiments demonstrate the feasibility of using MR to image probes selective for hsp90 in vivo, MR imaging of these compounds in cancer models should demonstrate the effectiveness of this strategy for improving diagnostic sensitivity and specificity.

Molecular probes and MR pulse sequences are tested in xenograft models of prostate, breast, and brain cancer. Imaging sequences and dosing regimens are optimized in vivo. Models are screened initially for hsp90 expression, as has been performed in brain xenografts (see FIG. 7), which shows up to 9-fold increased expression of hsp90 in some tumor lines.

In some embodiments, the presently disclosed subject matter provides for optimization of small molecule magnetic resonance imaging of hsp90 expression in tumor cells by investigating the imaging characteristics of a panel of small molecules with high selectivity but varying affinities for hsp90. While applicants do not wish to be bound by any particular theory of operation, it is possible that small molecules with high selectivity, but lower affinity and mid-range liphophilicity, compartmentalize within tumor cells expressing a high concentration of hsp90, but show little-to-no accumulation in normal cells with constitutively low expression of hsp90. Lower affinity, but high avidity, small molecules are thus anticipated to improve signal-to-noise ratios and further reduce toxicity.

A long-standing tenet of drug and imaging probe development holds that achieving high affinity for molecular targets is paramount. However, many useful molecular targets that are highly expressed in diseased cells are also expressed at some lower, basal level in normal cells. When high affinity drugs or imaging probes are administered in vivo, they often distribute in both normal cells and tissue as well diseased cells, producing unwanted binding and toxicity and reducing target to background ratio.

To address this shortcoming, disclosed herein is the discovery that lower affinity molecules, which are still highly selective for their protein target, do not distribute and accumulate in normal cells where protein targets are low in copy, but sequester in target cells where targets are high in copy through rebinding phenomenon. Low affinity small molecules which accumulate in compartments (e.g., intracellular) through rebinding phenomenon can be said to have high avidity for their target in the appropriate setting—that is, high localized concentrations of protein target. The phenomenon of probe-target rebinding has received prior treatment in the experiments disclosed in Frost & Wagner, 1984, which addressed the pharmacokinetic observation that in regions of the brain where opiate receptor densities are high, ligands (labeled drug and PET imaging probes) clear from the brain more slowly that in regions of the brain where the receptors are absent or in lower copy. Drawing on their theoretical treatment and empirical studies, one can predict the accumulation of small molecules in compartments with high target copy despite possessing low affinity for their intended target.

The imaging properties of a panel of highly selective small molecules with varying affinity for hsp90 are thus disclosed. Small molecules with micromolar affinity and midrange lipophilicity are expected to demonstrate favorable biodistribution properties, and therefore optimal signal-to-noise ratio, for imaging tumors in vivo.

The presently disclosed subject matter serves an unmet need for new non-invasive, non-radioactive diagnostic visualization of cancer and precancerous lesions currently undetectable using conventional imaging methods. Additionally, the disclosed subject matter allows for the strengths of medical chemistry to be directly applied to the development of novel imaging agents and is likely to have broad impact within radiological sciences in general.

### REFERENCES

The references listed below as well as all references cited in the specification are incorporated herein by reference to the extent that they supplement, explain, provide a background for, or teach methodology, techniques, and/or compositions employed herein.
Aime et al. (2002a) Angew Chem, Intl Ed 2002 41:4334.
Aime et al. (2002b) J Am Chem Soc 124:9364.
Aime et al. (2002c) Magn Reson Med 47:639.
Balaban & Ceckler (1992) Magn Reson Quart 8:116-137.
Donahue (2002) Moul J. Curr Urol Rep 3:215-221.
Erlichman et al. (2001) Proc AACR 42:A4474.
Eshalai (2003) Mag Res Med 49:755-759.
Freireich et al. (1966) Cancer Chemother Rep 50:219-244.
Frost & Wagner (1984) Brain Res 305:1-11.
Gaura et aL (2004) Neuroradiol 46:2005.
Goffeney et al. (2001) J Am Chem Soc 123:8628-8629.
Graves et al. (2002) Mol Pharm 62:1364-1372.
Grenert et al. (1997) J Biol Chem 272:23843-23850.
Guivel-Scharen et al. (1998) J Magn Reson 133:36-45.
Hartmann et al. (1997) Intl J Cancer 70:221-229.
Henkelman et al. (2001) NMR Biomed 14:57-64.
Hsieh & Balaban (1987) J Mag Res 74:574.
Mehta et al. (1996) Am Neuroradiol 17:105i.
McFarland et al. (1988) Mag Reson Imag 6:507.
Miller et al. (1994) Cancer Res 54:2724-2730.
Mimnaugh et al. (1996) J Biol Chem 271:22796-22801.
Misra et al. (2002) J Biol Chem 277:42082-42087.
Muise-Heimericks et al. (1998) J Biol Chem 273:29864-29872.
Newby & Greenbaum (2002) Proc Natl Acad Sci USA 99:12697-12702.
Ozsarlak et al. (2004) Eur Radiol 14:2067.
PCT International Patent Application Publication No. WO 2006/091963.
Sauer et al. (2002) Strahlenther Onkol 178:123-133.
Scheibel et al. (1999) Proc Natl Acad Sci USA 96:1297-1302.
Schneider et al. (1996) Proc Natl Acad Sci USA 93:14536-14541.
Schnur et al. (1995) J Med Chem 38:3806-3812.
Schulte et al. (1995) J Biol Chem 270:24585-24588.
Schulte et al. (1997) Biochem Biophys Res Commun 239:655-659.
Segnitz & Gehring (1997) J Biol Chem 272:18694-18701.
Sepp-Lorenzino et al. (1995) J Biol Chem 270:16580-16587.
Smith et al. (1995) Mol Cell Biol 15:6804-6812.
Snoussi et al. (2003) Mag Reson Med 49:998.
Stark & Bradley (eds.) (1991) Magnetic Resonance Imaging, C. V. Mosby Company, St. Louis, Mo., United States of America, pp. 204-218.
Strik et al. (2000) Anticancer Res 20:4457-4462.
Swanson (1998) J Magn Reson 135:248-255.
Stebbins et al. (1997) Cell 89:239-250.
United States Patent Application Publications 20050059881; 20060211737.
U.S. Pat. Nos. 5,050,609; 5,159,270; 6,943,033.
Van Zijl et al. (2003) Magn Reson Med 49:440.
Vasilevskaya et al. (1999) Cancer Res 59:3935-3940.
Ward et al. (2000) J Magn Reson 143:79-87.
Whitesell & Lindquist (2005) Nature Rev Cancer 10:761-772.
Whitesell et al. (1994) Proc Natl Acad Sci USA 91:8324-8328.
Wolff& Balaban (1990) J Magn Reson 86:164.
Zellweger et al. (2005) Intl J Cancer 113:619-628.
Zhang et al. (2002) Angew Chem, Intl Ed 41:1919.
Zhang et al. (2003a) Arc Chem Res 36:783.
Zhang et al. (2003b) J Am Chem Soc 125:15288-15289.
Zheng et al. (2001) Scand J Immunol 53:40-48.
Zhou et al. (2003) Nature Med 9:1085.
Zhou et al. (2004) Magn Reson Med 51:945.

It will be understood that various details of the presently disclosed subject matter can be changed without departing from the scope of the present disclosure. Furthermore, the foregoing description is for the purpose of illustration only, and not for the purpose of limitation.

What is claimed is:

1. A contrast enhancement agent useful for providing a visible image of a biological sample, the contrast enhancement agent comprising a small molecule ligand that binds to heat shock protein 90 (hsp90).

2. The contrast enhancement agent of claim 1, wherein the small molecule ligand comprises an indoline moiety.

3. The contrast enhancement agent of claim 1, wherein at least one atom present in the contrast enhancement agent is a resonant nucleus.

4. The contrast enhancement agent of claim 3, wherein the resonant nucleus is selected from the group including but not limited to ¹³C, ²³Na, ¹⁹F, ¹⁵N, ¹⁷O, and ³¹P.

5. The contrast enhancement agent of claim 1, further comprising a pharmaceutically acceptable diluent or excipient.

6. The contrast enhancement agent of claim 5, wherein the pharmaceutically acceptable diluent or excipient is pharmaceutically acceptable for use in a human.

7. An imaging composition comprising a small molecule ligand that binds to heat shock protein 90 (hsp90) and a pharmaceutically acceptable diluent or excipient.

8. The imaging composition of claim 7, wherein the pharmaceutically acceptable diluent or excipient is pharmaceutically acceptable for use in a human.

9. A method of non-invasively generating an image of a target tissue, the method comprising:
(a) providing a contrast enhancement agent comprising a small molecule that binds to a biomolecule present in the target tissue;
(b) introducing the contrast enhancement agent into the target tissue; and
(c) scanning the target tissue using magnetic resonance imaging, whereby an image of the target tissue is non-invasively generated.

10. The method of claim 9, wherein the imaging agent is disposed in a pharmaceutically acceptable diluent.

11. The method of claim 9, wherein the target tissue comprises a plurality of cells that overexpress hsp90.

12. The method of claim 9, wherein the target tissue is disposed in a subject.

13. The method of claim 12, wherein the target tissue is selected from the group including but not limited to an inflammatory lesion, a tumor, a pre-neoplastic lesion, a neoplastic cell, a pre-neoplastic cell, and a cancer cell.

14. The method of claim 13, wherein the tumor is selected from the group including but not limited to a primary tumor, a metastasized tumor, and a carcinoma.

15. The method of claim 13, wherein the target tissue is selected from the group including but not limited to a prostate tumor, a breast tumor, a uterine tumor, a melanoma, a glioma, a bladder carcinoma, a laryngeal carcinoma, a salivary gland tumor, and a leukemia.

16. The method of claim 12, wherein the subject is a mammal.

17. The method of claim 16, wherein the mammal is a human.

18. The method of claim 9, wherein the magnetic resonance imaging comprises magnetization/saturation transfer MRI.

19. The method of claim 9, wherein the biomolecule is an hsp90 polypeptide.

20. The method of claim 19, wherein the hsp90 polypeptide is a human hsp90 polypeptide.

21. The method of claim 9, wherein the introducing is by a route selected from the group including but not limited to oral, peroral, buccal, enteral, pulmonary, rectal, vaginal, nasal, lingual, sublingual, intravenous, intraarterial, intracardial, intramuscular, intraperitoneal, transdermal, intracranial, intracutaneous, subcutaneous, ocular, via an implant, and via a depot injection.

22. A method for monitoring a response to a therapy in a target tissue in a subject, the method comprising comparing a plurality of MRI images of a target tissue in the subject, wherein:
(a) a first subset of the plurality of MRI images are images of the target tissue generated prior to administering the therapy to the subject; and
(b) a second subset of the plurality of MRI images are images of the target tissue generated at a time subsequent to administering the therapy to the subject and at which a response to the therapy is expected;
(c) and further wherein the plurality of MRI images are generated by a method comprising:
(i) providing a contrast enhancement agent comprising a small molecule that binds to a biomolecule present in the target tissue;
(ii) introducing the contrast enhancement agent into the target tissue; and
(iii) scanning the target tissue using magnetic resonance imaging, whereby a visible image of the target tissue is non-invasively generated.

23. The method of claim 22, wherein the target tissue comprises a plurality of cells that overexpress hsp90.

24. The method of claim 22, wherein the target tissue is disposed in a subject.

25. The method of claim 22, wherein the target tissue is selected from the group including but not limited to an inflammatory lesion, a tumor, a pre-neoplastic lesion, a neoplastic cell, a pre-neoplastic cell, and a cancer cell.

26. The method of claim 25, wherein the tumor is selected from the group including but not limited to a primary tumor, a metastasized tumor, and a carcinoma.

27. The method of claim 25, wherein the target tissue is selected from the group including but not limited to a prostate tumor, a breast tumor, a uterine tumor, a melanoma, a glioma, a bladder carcinoma, a laryngeal carcinoma, a salivary gland tumor, and a leukemia.

28. The method of claim 22, wherein the subject is a mammal.

29. The method of claim 28, wherein the mammal is a human.

30. The method of claim 22, wherein the magnetic resonance imaging comprises magnetization/saturation transfer MRI.

31. The method of claim 22, wherein the biomolecule is an hsp90 polypeptide.

32. The method of claim 31, wherein the hsp90 polypeptide is a human hsp90 polypeptide.

33. A method of selecting a therapy for a subject, the method comprising:
(a) providing to the subject a contrast enhancement agent comprising a small molecule that binds to a biomolecule present in a target tissue in the subject;
(b) introducing the contrast enhancement agent into the target tissue; and
(c) scanning the target tissue using magnetic resonance imaging to determine if the target tissue overexpresses the biomolecule compared to a standard, whereby overexpression or lack of overexpression of the biomolecule in the target tissue provides a basis for selecting a therapy for the subject.

34. The method of claim 33, wherein the subject is a human.

35. The method of claim 33, wherein the biomolecule comprises an hsp90 polypeptide.

36. The method of claim 33, wherein the target tissue comprises a plurality of cells that overexpress hsp90.

37. The method of claim 36, wherein the target tissue is selected from the group including but not limited to an inflammatory lesion, a tumor, a pre-neoplastic lesion, a neoplastic cell, a pre-neoplastic cell, and a cancer cell.

38. The method of claim 37, wherein the tumor is selected from the group including but not limited to a primary tumor, a metastasized tumor, and a carcinoma.

39. A method for delineating a boundary between a diseased cell and a non-diseased cell in a tissue in a subject, the method comprising:
(a) providing to the subject a contrast enhancement agent comprising a small molecule that binds to a biomolecule that is differentially expressed in the diseased cell and the non-diseased cell in the tissue;
(b) introducing the contrast enhancement agent into the diseased cell and into the non-diseased cell in the tissue; and
(c) scanning the tissue using magnetic resonance imaging
whereby a boundary between a diseased cell and a non-diseased cell in the tissue in the subject is delineated.

40. The method of claim 39, wherein the diseased cell is selected from the group including but not limited to a tumor cell, a pre-neoplastic cell, a neoplastic cell, and a cancer cell.

41. The method of claim 40, wherein the boundary comprises a boundary between a tumor cell or a cancer cell and a normal cell in tissue surrounding the tumor cell or the cancer cell.

42. A method for assessing the degree to which a target tissue has been removed from a subject, the method comprising imaging a region in which the target tissue was present in the subject to assess for the degree to which cells that overexpress a biomolecule of interest have been removed from and/or remain in the target tissue.

43. The method of claim 42, wherein the target tissue comprises a tumor or cancer cell that overexpresses hsp90.

44. The method of claim 43, wherein the subject is a human.
